(19) United States
(12) Patent Application Publication
(10) Pub. No.: US 2009/0202613 A1
(43) Pub. Date: Aug. 13, 2009

(54) Nutritionally Enhanced Therapeutic Preventative Food Supplement and Method of Making Same

(75) Inventor: Ben Tabatchnick, Somerset, NJ (US)

Correspondence Address: BLANK ROME LLP, ONE LOGAN SQUARE, PHILADELPHIA, PA, 19103, US

(73) Assignee: Tabatchnick Fine Foods, Inc., Somerset, NJ (US)

(21) Appl. No.: 12/368,342

(22) Filed: Feb. 10, 2009

Related U.S. Application Data
(60) Provisional application No. 61/027,653, filed on Feb. 11, 2008.
(60) Provisional application No. 61/044,709, filed on Apr. 14, 2008.

(51) Int. Cl. A61K 9/00; A61K 38/16

(52) U.S. Cl. 424/439; 514/6

(57) ABSTRACT

The present invention is directed to a nutritionally enhanced therapeutic preventative food supplement which includes one or more food ingredients, a mineral and vitamin mix component, a base coating component, and an immune system potentiate, wherein the immune system potentiate is not denatured. The present invention includes a process to make an enhanced therapeutic food supplement and a method to potentiate the immune system of a mammal in need thereof.

### CROSS-REFERENCE TO RELATED APPLICATIONS

This application claims priority to U.S. Provisional application No. 61/027,653, filed Feb. 11, 2008 and U.S. Provisional application No. 61/044,709, filed Apr. 14, 2008, both of which are incorporated herein in their entirety.

### FIELD OF THE INVENTION

This invention relates generally to a ready to use nutritionally enhanced therapeutic preventative food supplement having an immune system potentiate and process for making same.

### BACKGROUND OF THE INVENTION

As the world population grows, efforts are continually occurring to feed the moderate and severely malnourished and to combat diseases which are associated with these maladies. As reported by the United Nations, approximately 800 million people in the developing world are chronically undernourished. Malnutrition, measles, diarrhea and dehydration remain the four leading causes in the death of 6 million children under the age of 5 each year, as reported by the World Health Organization. Moreover, an additional 3 million people in sub-Saharan Africa contracted HIV/AIDS in 2001, bringing the number of infected people in the region to 28.5 million; an estimated 11 million children have been orphaned by the disease.

Ready to use therapeutic foods (RUTFs) enriched with nutraceutical and immune potentiating components are important to combat not only malnutrition but also prevention of diseases including HIV/AIDS, polio, cholera and dysentery. Important to the effectiveness of RUTFs are that they do not require water or mixing or any other type of preparation. In areas wherein malnutrition and HIV/AIDS are prominent, additional preparation and storage issues exist which cause problems in dosage and sanitary conditions for mixing components.

Incorporation of an immune system potentiate into a food supplement allows for a variety of properties. Proteins capable of complexing iron, such as lactoferrin and transferrin have many biological properties and have been found in baby formula for over 50 years. Specifically, lactoferrin is a part of the innate immune system and provides a first line defense against invading bacteria, viruses, parasites, yeasts, fungi and toxins. Lactoferrin influences the intestinal micro flora by inhibiting pathogenic bacteria and promoting the growth of beneficial organisms. Lactoferrin may be of bovine, equine or ovine origin, or else may be obtained synthetically or via the recombinant pathway. Thus, its use in the food supplement field, and in the pharmaceutical field is recognized as discussed in U.S. Pat. Nos. 6,984,624, 6,991,824, and 5,576,299, incorporated herein in relevant part.

It is desired to provide a food supplement which includes an immune potentiating component to combat opportunistic infection and addresses issues of malnutrition in a ready-to-use form that is chemically stable and cost effective.

### BRIEF SUMMARY OF THE INVENTION

In a first embodiment, the present invention is directed to a nutritionally enhanced therapeutic preventative food supplement which includes one or more food ingredients, a mineral and vitamin mix component, a base coating component, and an immune system potentiate, wherein the immune system potentiate is not denatured.

The first embodiment of the present invention is the incorporation of an immune system potentiate, such as lactoferrin, having a purity of at least 99% and enhanced with nutrients. Specifically, the inclusion of natural milk proteins which have been purified and enriched with beneficial proteins so as to invoke the greatest immune response. Inclusion of the lactoferrin protein for use as an immune system potentiate of the present invention is included in a state wherein it is not denatured.

The present invention contains an immune potentiate of about 0.04 to about 0.06 percent by weight of the food supplement, a mineral and vitamin mix of about 0.8 to about 3.0 percent by weight of the food supplement, a coating which is at least 50 percent and no greater than 75 percent by weight of the food supplement, and food ingredients which are less than 50 percent by weight of the food supplement.

The one or more food ingredients include carbohydrates and lipids. The carbohydrates are selected from a group which includes sucrose, glucose, fructose, maltodextrin, dry milk products, whey products, or flours made of corn, wheat, soy, oats, rice or potato. The milk product and the whey product are less than 2.0 percent by weight of the food supplement. The lipids are selected from the groups consisting of oils, fats or cholesterol and are at least 11.2 percent and no greater than 20.0 percent by weight of the food supplement. The fats include saturated fats, mono- and poly-unsaturated fats.

The coating component includes a lipid rich substance which may be derived from oleaginous seeds such as peanut, cocoa beans, almonds, coconuts or pistachio nuts, sesame, soy, walnuts, almonds, sunflower seed, pumpkin seed, or they can be a mixture of various fats of vegetable origin.

In another embodiment, the present invention is directed to a nutritionally enhanced therapeutic preventative food supplement which contains no dairy based components. The food supplement includes one or more food ingredients, a base coating component, and an immune system potentiate, wherein the immune system potentiate is not denatured.

The supplement includes more than 2 percent and less than 15 percent of soy protein isolate, hydrolysate or concentrate, more than 20 percent and less than 50 percent of oleaginous paste, more than 12 percent and less than 30 percent vegetable oil, more than 12 percent and less than 25 percent sugar, more than 22 percent and less than 44 percent of filler, wherein the filler comprises maltodextrin or corn syrup solids or a combination of thereof. In addition, color, mono/diglycerides, vitamin mix and lactoferrin are added between at ranges of 0.01 to 3.0 percent. Lactoferrin is added at about 0.05 percent or about 50 milligrams per 100 grams of food supplement.

In another embodiment, the present invention is directed to a nutritionally enhanced therapeutic preventative food supplement which includes one or more food ingredients, a base coating component, and an immune system potentiate, wherein the immune system potentiate is not denatured. In this embodiment, the food supplement includes dairy based components such as nonfat dry milk and a whey protein isolate, hydrolysate or concentrate.

In this embodiment, the food supplement comprises less than 2 percent nonfat dry milk, less than 2 percent whey protein isolate, hydrolysate or concentrate, more than 50 percent and less than 75 percent of oleaginous paste, more than 12 percent and less than 20 percent vegetable oil, more than 12 percent and less than 25 percent sugar, more than 22 percent and less than 44 percent of filler, wherein the filler comprises maltodextrin. In addition, color, mono/diglycerides, vitamin mix and lactoferrin are added between at ranges of 0.01 to 3.0 percent. Lactoferrin is added at about 0.05% or about 50 milligrams per 100 grams of food supplement.

In another embodiment, the present invention is directed to a nutritionally enhanced therapeutic preventative food supplement which includes one or more food ingredients, a base coating component, and an immune system potentiate, wherein the immune system potentiate is not denatured. In this embodiment, the food supplement includes non-dairy based nonfat dry milk, a soy protein isolate, hydrolysate or concentrate, a whey isolate, hydrolysate or concentrate, or combination thereof, wherein the oleaginous paste of the previous embodiments is replaced with corn syrup so as prevent any allergic reaction to peanuts based ingredients.

The food supplement of this embodiment comprises a combination of components including less than 2 percent of nonfat dry milk, more than 1 percent and less than 8 percent of soy protein isolate, hydrolysate or concentrate, more than 1 percent and less than 8 percent of whey protein isolate, hydrolysate or concentrate, more than 20 percent and less than 50 percent of corn syrup, more than 15 percent and less than 50 percent vegetable oil, more than 6 percent and less than 22 percent sugar. In addition, color, mono/diglycerides, vitamin mix and lactoferrin are added between at ranges of 0.01 to 3.0 percent. Lactoferrin is added at about 0.05% or about 50 milligrams per 100 grams of food supplement.

The food supplement has a minimum of 10 milligrams of and no more than 1500 milligrams lactoferrin and can be stored at −10° F. to 110° F. for 2 years without decomposing as in the previous two embodiments.

In another embodiment, the invention is directed to a therapeutic preventative food supplement product prepared by a process including the steps of heating oil to 145 degrees Fahrenheit and blending in the first 50% of the total amount of a base coating component to be blended into the food supplement. Thereafter, the other components are added that include a mineral component, an immune system potentiate, most commonly, a substantially pure form of lactoferrin, which is not denatured and is enhanced with nutrients, and at least one food ingredient is added. A second 50% of the total amount of a coating component is then blended in.

In another embodiment, the present invention is directed to a method of potentiating the host immune system in an immunocompromised mammal in need thereof. The method includes consuming a therapeutic preventative food supplement by the immunocompromised mammal in an amount effective to potentiate the host immune system against opportunistic microorganisms, wherein the immunocompromised condition is caused by viral infection by opportunistic microorganisms, such as protozoa or fungi, or parasitic infections. The food supplement includes one or more food ingredients, a mineral component, a base coating component, and an immune system potentiate, wherein the immune system potentiate is not denatured.

### DETAILED DESCRIPTION OF THE INVENTION

Certain terminology is used herein for convenience only and is not to be taken as a limitation on the present invention. The terminology includes the words specifically mentioned, derivatives thereof and words of similar import. The embodiments discussed herein are not intended to be exhaustive or to limit the invention to the precise form disclosed. These embodiments are chosen and described to best explain the principle of the invention and its application and practical use and to enable others skilled in the art to best utilize the invention.

In the first embodiment, the present invention is directed to a nutritionally enhanced therapeutic preventative food supplement which includes one or more food ingredients, a mineral and vitamin mix component, a coating component, and an immune system potentiate, wherein the immune system potentiate is not denatured.

The present invention includes an immune system potentiate, such as lactoferrin having a purity of at least 99% and enhanced with nutrients. Specifically, natural milk proteins which have been purified and enriched with beneficial proteins are desired in the present invention so as to invoke the greatest immune response. Such products include Bioferrin® produced by Glanbia Nutritionals Inc. This response is effective against infection by opportunistic disease caused by various sources including viruses which cause AIDS. A “one-stop” supplement to combat malnutrition and HIV infection is key in prevention so as to ensure regular and repeat dosage.

Inclusion of the lactoferrin protein for use as an immune system potentiate is included in a state wherein it is not denatured. As is recognized by those of skill in the art, denaturizing of a protein will cause the protein to change shape and therefore become less effective or not effective when used. Typically, denaturizing occurs in the process of preparation of the food supplement and yields a less effective product. However, it is important to recognize that the present invention and method of preparation thereof (as described herein) allows the base coating to act as a preservative of the lactoferrin as the base coating is distributed substantially evenly (based on the process of preparation) for contact with each of the components of the food supplement including, most importantly, the lactoferrin which act as the immune system potentiate.

The food supplement of the present invention has a unique formula which will yield effective costs, an acceptable shelf life and provides high nutritional content to rehabilitate malnourished individuals. Specifically, the present invention contains an immune potentiate of about 0.04 to about 0.06 percent by weight of the food supplement, a mineral and vitamin mix is about 0.8 to about 3.0 percent by weight of the food supplement, a coating is at least 50 percent and no greater than 75 percent by weight of the food supplement, and food ingredients are less than 50 percent by weight of the food supplement.

The one or more food ingredients include carbohydrates and lipids. The carbohydrates are selected from a group which includes sucrose, glucose, fructose, maltodextrin, dry milk products, whey products, or flours made of corn, wheat, soy, oats, rice or potato. The maltodextrin used primarily as a filler in the food supplement is more than 21 percent and less than 44 percent by weight of the supplement, while sugars, such as sucrose, glucose and fructose or combinations thereof, are greater the 12.0 percent and less than 25 percent by weight of the supplement.

The milk product and the whey products are less than 2.0 percent by weight of the food supplement. This low amount of milk product and whey product content reduces the cost of the final food supplement product and therefore allows greater distribution of the food supplement to those in need. In addition, the low milk content allows greater stability from decomposition and bacteria attack.

The lipids are selected from the group consisting of oils, fats or cholesterol. The fats include saturated fats, mono- and poly-unsaturated fats. The lipids are at least 11.2 percent and less than 20.0 percent by weight of the food supplement and include, by way of example, soybean, corn, cottonseed, palm kernel, olive, canola or other “bean or seed type” vegetable oil.

In this embodiment the coating component comprises a lipid rich substance which may be derived from oleaginous seeds of peanut, cocoa beans, almonds, coconuts, pistachio nuts, sesame, soy, walnuts, almonds, sunflower seed, pumpkin seed, or mixture thereof, or they can be a mixture of various fats of vegetable origin. The use of peanut butter in the present invention allows greater stability of the food supplement and acts to preserve the other components of the food supplement.

Additional substances such as color enhancers and flavors may be added as desired so long as they do not affect shelf life, storage or overall stability of the supplement. In addition, mono/diglycerides may be added for completion of mixing. Furthermore, stabilizers can be added to the food supplement for increased shelf life. These stabilizers include Durem #207, produced by Loders Croklaan, Inc. These stabilizers are less than 1 percent by weight of the food supplement.

The therapeutic preventative food supplement is ready for consumption directly from a package made of foil, paper and/or plastic. Each 3.2 ounce portion of the food supplement of the present embodiment contains at least 50 milligrams of an immune potentiate and develops an osmolality of greater than 100 mOsm/kg (milliosmoles per kilogram) after immersion in four times its own volume in water. This property exists until agitation. Most particularly, the present embodiment has a osmolality of greater than 100 but less than 150 mOsm/kg after immersion in four times its own volume in water. Based on the composition of the food supplement as packaged, no adding of water or additional moisture, cooking or refrigeration is necessary. This is important based on the use of the food supplement of the present invention in environments wherein water is not pure and is contaminated with bacterial, or in climate of high temperatures which effect food storage.

Additional embodiments of the invention will now be described wherein like components will have the same definition.

In another embodiment, the present invention is directed to a nutritionally enhanced therapeutic preventative food supplement which contains no dairy based components. The food supplement includes one or more food ingredients, a base coating component, and an immune system potentiate, wherein the immune system potentiate is not denatured.

Protein is derived from a soy protein isolate, hydrolysate or concentrates which gives a higher concentration of protein than soy protein obtained from natural foods. This form of soy protein gives two beneficial effects; 1) it gives a greater amount of protein in a concentrated form; thereby maintaining an accepted daily serving criteria, and 2) eliminates the need for components in the food supplement that may allow bacteria growth and effect storage issues including environment and shelf life.

In this embodiment, the supplement includes more than 2 percent and less than 15 percent of soy protein, more than 20 percent and less than 50 percent of oleaginous paste, more than 12 percent and less than 30 percent vegetable oil, more than 12 percent and less than 25 percent sugar, and more than 22 percent and less than 44 percent of filler, wherein the filler comprises maltodextrin or corn syrup solids or a combination of thereof. In addition, color, mono/diglycerides, vitamin mix and lactoferrin are added between at ranges of 0.01 to 3.0 percent. Lactoferrin is added at about 0.05 percent or about 50 milligrams per 100 grams of food supplement.

The food supplement of this embodiment has a minimum of 50 milligrams of lactoferrin per 3.2 ounce serving and can be stored at −10° F. to 110° F. for 2 years without decomposing. These attributes are based on the concentrated form of protein used in preparation of the food supplement and because no water is added to the formulation. These attributes are very beneficial in obtaining the objectives regarding world hunger and prevention of disease, most specifically, AIDS.

In another embodiment, the present invention is directed to a nutritionally enhanced therapeutic preventative food supplement which includes one or more food ingredients, a base coating component, and an immune system potentiate, wherein the immune system potentiate is not denatured. In this embodiment, the immune system potential is derived from dairy based components such as nonfat dry milk and a whey protein isolate, hydrolysate or concentrate.

In this embodiment, the food supplement includes less than 2 percent nonfat dry milk, less than 2 percent whey protein isolate, hydrolysate or concentrate, more than 50 percent and less than 75 percent of oleaginous paste, more than 12 percent and less than 20 percent vegetable oil, more than 12 percent and less than 25 percent sugar, and more than 22 percent and less than 44 percent of filler, wherein the filler includes maltodextrin, more than 12 percent and less than 25 percent sugar. In addition, color, mono/diglycerides, vitamin mix and lactoferrin are added between at ranges of 0.01 to 3.0 percent. Lactoferrin is added at about 0.05% or about 50 milligrams per 100 grams of food supplement.

The food supplement has a minimum of 50 milligrams of lactoferrin and can be stored at −10° F. to 110° F. for 2 years without decomposing as in the previous embodiment.

In another embodiment, the present invention is directed to a nutritionally enhanced therapeutic preventative food supplement which includes one or more food ingredients, a coating component and an immune system potentiate, wherein the immune system potentiate is not denatured. In this embodiment, the immune system potential is derived from nonfat dry milk, a soy protein isolate, hydrolysate or concentrate, a whey isolate, hydrolysate or concentrate or combination thereof, but does not contain any peanut based component so as to avoid the possibility of allergic reaction.

The food supplement of this embodiment includes less than 2 percent of nonfat dry milk, more than 1 percent and less than 8 percent of soy protein isolate, hydrolysate or concentrate, more than 1 percent and less than 8 percent of whey protein isolate, hydrolysate or concentrate, more than 20 percent and less than 50 percent of corn syrup as a filler, more than 12 percent and less than 30 percent vegetable oil, more than 12 percent and less than 25 percent sugar. In addition, color, mono/diglycerides, vitamin mix and lactoferrin are added between at ranges of 0.01 to 3.0 percent. Lactoferrin is added at about 0.05% or about 50 milligrams per 100 grams of food supplement.

The food supplement has a minimum of 50 milligrams of lactoferrin, and can be stored at −10° F. to 100° F. for 2 years without decomposing as in the previous two embodiments.

In another embodiment, the invention is directed to a therapeutic preventative food supplement product prepared by a process including the steps of heating oil to 145 degrees Fahrenheit and blending in a first 50% of the total amount of a base coating component to be blended into the food supplement. All other components for each embodiment, e.g. the mineral component, an immune system potentiate and food ingredients are than added, and thereafter, the second 50% of the total amount of base coating component is added into the food supplement. The product is cooled to 100° F. and packaged in a form fill seal machine utilizing two different laminates that are heat sealed together to form a pouch. The product is injected into each pouch and hermetically sealed.

This process results in the food supplement having; 1) substantially equal distribution of the components, 2) removal of substantially all water for each component, and therefore the resultant food supplement has a minimal amount of water (less than 1.0 percent), and 3) ensures all yeast, mold or other bacteria are eliminated so as not to contaminate the resultant food supplement product. Though it is appreciated some moisture may exist in the product, the absence of water, and equal distribution of the components with the based coating component, both attributed to the heating and mixing with oil, results in the extended shelf life of the product. Therefore, costs are reduced based on the reduction or elimination due to spoilage or contamination of the food supplement product while in storage at extreme conditions (as discussed herein).

Since lactoferrin denatures at a temperature of about 180° F. or higher, the process of the present invention uses a temperature lower so as to ensure the lactoferrin is not denatured and is chemically active in the food supplement.

Each of the food supplement embodiments described herein is prepared by the this process which is simple, and therefore, limits costs of the food supplement product so as to allow a large distribution area while being cost effective.

In another embodiment, the present invention is directed to a method of potentiating the host immune system in an immunocompromised mammal in need thereof. The method includes consuming a therapeutic preventative food supplement by an immunocompromised mammal in an amount effective to potentiate the host immune system against opportunistic microorganisms, wherein the immunocompromised condition is caused by viral infection by opportunistic microorganisms, such as protozoa or fungi, or parasitic infections. The food supplement includes one or more food ingredients, a base coating component, and an immune system potentiate, as described and defined in the previous embodiments herein. The immune potentiate is a substantially pure form of lactoferrin enhanced with nutrients which has not been denatured, delivered in a minimum dose of 50 milligrams daily. The method of this embodiment could include any of the embodiments discussed herein.

Examples of the embodiments will now be presented in detail; the components are described by the percentage of the total weight of the food supplement.

### EXAMPLES

### Example 1

A nutritionally enhanced therapeutic preventative food supplement:

| | |
|---|---|
| Peanut Butter | 50.0% |
| Maltodextrin | 22.0% |
| Sugar granulated | 12.0% |
| Soybean Oil | 12.0% |
| Non Fat dry milk | 1.5% |
| Whey protein | 1.0% |
| Durem #207 | .5% |
| Minerals and Vitamin Mix | 1.0% |
| Bioferrin ® | 0.05% |

The supplement is prepared by adding half of the Peanut Butter to the 12% of the Soybean Oil and heating to 145° F. When the Peanut Butter is completely mixed with the heated oil, the maltodextrin, sugar, Non Fat dry milk, Whey protein, Durem #207 and Minerals and Vitamin Mix are added. Upon mixing, a second half of the Peanut Butter is added and mixed. The product is cooled to 100° F. and packaged in a form fill seal machine utilizing two different laminates that are heat sealed together to form a pouch. The product is injected into each pouch and hermetically sealed.

### Example 2

A non-dairy nutritionally enhanced therapeutic preventative food supplement:

| | |
|---|---|
| Peanut Butter | 28.28% |
| Maltodextrin | 23.06% |
| Sugar granulated | 15.25% |
| SPI Supro 660 | 6.6% |
| Vegetable oil | 19.31% |
| Color | 0.2% |
| Flavor | 0.24% |
| Mono & Diglycerides | 0.58% |
| Minerals and Vitamin Mix | 2.85% |
| Lactoferrin | 0.05% |

The supplement is prepared by adding half of the Peanut Butter to the 20% of the Vegetable Oil and heating to 145° F. When the Peanut Butter is completely mixed with the heated oil, the maltodextrin, sugar, and soy isolate, color, mono/diglycerides, vitamin mix and lactoferrin are added. Upon mixing, a second half of the Peanut Butter is added and mixed. The product is cooled to 100° F. and packaged in a form fill seal machine utilizing two different laminates that are heat sealed together to form a pouch. The product is injected into each pouch and hermetically sealed.

### Example 3

A nutritionally enhanced therapeutic preventative food supplement of dairy based components:

| | |
|---|---|
| Peanut Butter | 27.92% |
| Maltodextrin | 25.46% |
| Sugar granulated | 15.45% |
| WPI Provon 190 | 6.6% |
| Non Dairy Milk | 1.36% |
| Vegetable oil | 19.31% |
| Color | 0.2% |
| Flavor | 0.24% |
| Mono & Diglycerides | 0.58% |
| Minerals and Vitamin Mix | 2.85% |
| Lactoferrin | 0.05% |

The supplement is prepared by adding half of the Peanut Butter to the 12.5% of the Soybean Oil and heating to 145° F. When the Peanut Butter is completely mixed with the heated oil, the maltodextrin, sugar, Non Fat dry milk, Whey protein, color, mono/diglycerides, vitamin mix and lactoferrin are added. Upon mixing, a second half of the Peanut Butter is added and mixed. The product is cooled to 100° F. and packaged in a form fill seal machine utilizing two different laminates that are heat sealed together to form a pouch. The product is injected into each pouch and hermetically sealed.

### Example 4

A non-dairy and no peanut nutritionally enhanced therapeutic preventative food supplement:

| | |
|---|---|
| Corn Syrup Solids 20 DE | 39.07% |
| Sugar granulated | 8.26% |
| SPI Supro 660 | 15.36% |
| Vegetable oil | 33.53% |
| Color | 0.20% |
| Flavor | 0.24% |
| Mono & Diglycerides | 0.48% |
| Flour Salt | 0.2% |
| Minerals and Vitamin Mix | 2.77% |
| Lactoferrin | 0.03% |

The supplement is prepared by adding half of the Peanut Butter to the 12.5% of the Soybean Oil and heating to 145° F. When the Corn Syrup is completely mixed with the heated oil, sugar, Soy protein isolate, flour salt, color, mono/diglycerides, vitamin mix and lactoferrin are added. Upon mixing, a second half of the Corn Syrup is added and mixed. The product is cooled to 100° F. and packaged in a form fill seal machine utilizing two different laminates that are heat sealed together to form a pouch. The product is injected into each pouch and hermetically sealed.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A nutritionally enhanced therapeutic preventative food supplement comprising:
one or more food ingredients;
a base coating component; and
an immune system potentiate, wherein the immune system potentiate is not denatured.

2. The food supplement of claim 1, wherein the immune system potentiate is lactoferrin.

3. The food supplement of claim 2, wherein the immune potentiate is substantially pure form of lactoferrin enhanced with nutrients.

4. The food supplement of claim 3, wherein the immune potentiate is about 0.04 to about 0.06 percent by weight of the food supplement.

5. The food supplement of claim 4, further comprising a mineral and vitamin mix component wherein the mineral and vitamin mix is about 0.8 to about 3.0 percent by weight of the food supplement.

6. The food supplement of claim 5, wherein the coating is at least 50 percent and no greater than 75 percent by weight of the food supplement.

7. The food supplement of claim 6, wherein the food ingredients are less than 50 percent by weight of the food supplement.

8. The food supplement of claim 7, wherein 3.2 ounces of food supplement contain at least 50 milligrams of lactoferrin, wherein the food supplement develops an osmolality of greater than 100 mOsm/kg after immersion in four times its own volume in water.

9. The food supplement of claim 8, wherein the one or more food ingredients comprises carbohydrates and lipids.

10. The food supplement of claim 9, wherein lipids are at least about 11.2 percent and less than about 20.0 percent by weight of the food supplement.

11. The food supplement of claim 10, wherein the carbohydrates are selected from a group consisting of: sucrose, glucose, fructose, maltodextrin, dry milk products, whey products, or flours made of corn, wheat, soy, oats, rice or potato.

12. The food supplement of claim 11, wherein the milk product is less than 2.0 percent by weight of the food supplement.

13. The food supplement of claim 10, wherein lipids are selected from the groups consisting of oils, fats or cholesterol.

14. The food supplement of claim 7, wherein the base component comprises a lipid rich substance derived from oleaginous seeds comprising peanut, cocoa beans, almonds, coconuts, pistachio nuts or mixture thereof, or a mixture of various fats of vegetable origin.

15. The food supplement of claim 5, wherein the therapeutic preventative food supplement is ready for consumption directly from a package.

16. The food supplement of claim 15, wherein the food supplement contains no dairy based components and no water added.

17. The food supplement of claim 16, wherein the food component comprises a soy protein isolate, hydrolysate or concentrate.

18. The food supplement of claim 17, wherein the food supplement comprises more than 2 percent and less than 15 percent of soy protein isolate, hydrolysate or concentrate, more than 20 percent and less than 50 percent of oleaginous paste, more than 12 percent and less than 30 percent vegetable oil, more than 22 percent and less than 44 percent of filler, wherein the filler comprises maltodextrin or corn syrup solids or a combination of thereof, more than 12 percent and less than 25 percent sugar.

19. The food supplement of claim 18, wherein 100 grams of food supplement has a minimum of 50 milligrams of lactoferrin.

20. The food supplement of claim 19, wherein the food supplement can be stored at −10° F. to 110° F. for 2 years without decomposing.

21. The food supplement of claim 15, wherein the food supplement contains dairy based components, wherein the food supplement has no water added.

22. The food supplement of claim 21, wherein the food supplement comprises nonfat dry milk and a whey protein isolate, hydrolysate or concentrate.

23. The food supplement of claim 22, wherein the food supplement comprises less than 2 percent nonfat dry milk, less than 2 percent whey protein, more than 50 percent and less than 75 percent of oleaginous paste, more than 12 percent and less than 20 percent vegetable oil, more than 22 percent and less than 44 percent of filler, wherein the filler comprises maltodextrin, more than 12 percent and less than 25 percent sugar.

24. The food supplement of claim 23, wherein 100 grams of food supplement has a minimum of 50 milligrams of lactoferrin.

25. The food supplement of claim 24, wherein the food supplement can be stored at −10° F. to 110° F. for 2 years without decomposing.

26. The food supplement of claim 15, wherein the food supplement does not have any nut or seed based ingredients, wherein the food supplement comprises nonfat dry milk, a soy protein isolate, hydrolysate or concentrate, a whey isolate, hydrolysate or concentrate or combination thereof, wherein the food supplement has no water added.

27. The food supplement of claim 26, wherein the food supplement comprises less than 2 percent of nonfat dry milk, more than 1 percent and less than 8 percent of soy protein, more than 1 percent and less than 8 percent of whey protein, more than 20 percent and less than 50 percent of oleaginous paste, more than 12 percent and less than 30 percent vegetable oil, more than 22 percent and less than 44 percent of filler, wherein the filler comprises maltodextrin or corn syrup solids or a combination of thereof, more than 12 percent and less than 25 percent sugar.

28. The food supplement of claim 27, wherein the 100 grams of food supplement has a minimum of 50 milligrams of lactoferrin.

29. The food supplement of claim 28, wherein the food supplement can be stored at −10° F. to 110° F. for 2 years without decomposing.

30. A method of potentiating the host immune system in an immunocompromised mammal in need thereof, comprising: consuming a therapeutic preventative food supplement by the immunocompromised mammal in an amount effective to potentiate the host immune system against opportunistic microorganisms, wherein the immunocompromised condition is caused by viral infection, and wherein the opportunistic microorganisms are protozoa or fungi, or parasitic infections, wherein the food supplement comprises an immune system potentiate, wherein the immune system potentiate is not denatured.

31. The method of claim 30, wherein the food supplement further comprises:
one or more food ingredients; and
base coating component.

32. The method of claim 31, wherein the immune system potentiate is lactoferrin.

33. The method of claim 32, wherein the immune potentiate is a substantially pure form of lactoferrin enhanced with nutrients.

34. The method of claim 33, wherein the food supplement contains no dairy based components, wherein the food component comprises a soy protein isolate, hydrolysate or concentrate, wherein the food supplement has no water.

35. The method of claim 33, wherein the food supplement comprises dairy based components, wherein the food component comprises non-fat dry milk, and a whey protein isolate, hydrolysate or concentrate, wherein the food supplement has no water.

36. The method of claim 33, wherein the food supplement comprises a soy protein isolate, hydrolysate or concentrate, wherein the food supplement comprises non-fat dry milk, and a whey protein isolate, hydrolysate or concentrate, wherein the food supplement has no water added, wherein the food supplement contains no nut or seed products.

37. A therapeutic preventative food supplement product prepared by the process of comprising the steps of:
a. heating oil to 145 degrees Fahrenheit;
b. blending in a first 50 percent of the total amount of a base coating component to be blended into the food supplement;
c. adding a mineral component, an immune system potentiate and at least one food ingredient;
d. blending in the second 50 percent of the total amount of a coating component to be blended into the food supplement;
e. cooling the product to 100 degrees Fahrenheit; and
f. packaging the product within a pouch comprised of different laminates that are heat sealed together,
wherein the immune potentiate is lactoferrin.

38. The therapeutic preventative food supplement of claim 37, wherein the lactoferrin is a substantially pure form of lactoferrin which is not denatured and is enhanced with nutrients.

39. The method of claim 38, wherein the food supplement contains no dairy based components, wherein the food component comprises a soy protein isolate, hydrolysate or concentrate, wherein the food supplement has no water added.

40. The method of claim 38, wherein the food supplement comprises dairy based components, wherein the food component comprises non-fat dry milk, and a whey protein isolate, hydrolysate or concentrate, wherein the food supplement has no water added.

41. The method of claim 38, wherein the food supplement comprises a soy protein isolate, hydrolysate or concentrate, wherein the food supplement comprises non-fat dry milk, and a whey protein isolate, hydrolysate or concentrate, wherein the food supplement has no water, wherein the food supplement contains no nut or seed products.
